(19) United States
(12) Patent Application Publication
(10) Pub. No.: US 2009/0259091 A1
(43) Pub. Date: Oct. 15, 2009

(54) BONE CONDUCTION DEVICE HAVING A PLURALITY OF SOUND INPUT DEVICES

(75) Inventor: John L. Parker, Roseville (AU)

Correspondence Address: CONNOLLY BOVE LODGE & HUTZ LLP, 1875 EYE STREET, N.W., SUITE 1100, WASHINGTON, DC, 20006, US

(73) Assignee: COCHLEAR LIMITED, Lane Cove (AU)

(21) Appl. No.: 12/355,415

(22) Filed: Jan. 16, 2009

Related U.S. Application Data
(60) Provisional application No. 61/041,185, filed on Mar. 31, 2008.

(51) Int. Cl. A61F 11/00; H04R 25/00

(52) U.S. Cl. 600/25

(57) ABSTRACT

The present invention relates to a bone conduction device for enhancing the hearing of a recipient is provided. The bone conduction device may include a first sound input device configured to receive sound signals and generate a first electrical signal representative of the signal, a second sound input device configured to receive sound signals and generate a second electrical signal representative of the signal, electronic circuitry configured to select at least one of the first electrical signal and the second electrical signal, and an electronics module configured to generate a third electrical signal representing the sound signals based on at least of the first electrical signal and the second electrical signal.

### CROSS-REFERENCE TO RELATED APPLICATIONS

The present application claims the benefit of U.S. Provisional Patent Application No. 61/041,185; filed Mar. 31, 2008, which is hereby incorporated by reference herein.

### BACKGROUND

1. Field of the Invention

The present invention is generally directed to a bone conduction device, and more particularly, to a bone conduction device having a plurality of sound input devices.

2. Related Art

Hearing loss, which may be due to many different causes, is generally of two types, conductive or sensorineural. In many people who are profoundly deaf, the reason for their deafness is sensorineural hearing loss. This type of hearing loss is due to the absence or destruction of the hair cells in the cochlea which transduce acoustic signals into nerve impulses. Various prosthetic hearing implants have been developed to provide individuals who suffer from sensorineural hearing loss with the ability to perceive sound. One such prosthetic hearing implant is referred to as a cochlear implant. Cochlear implants use an electrode array implanted in the cochlea of a recipient to provide an electrical stimulus directly to the cochlea nerve, thereby causing a hearing sensation.

Conductive hearing loss occurs when the normal mechanical pathways to provide sound to hair cells in the cochlea are impeded, for example, by damage to the ossicular chain or ear canal. Individuals who suffer from conductive hearing loss may still have some form of residual hearing because the hair cells in the cochlea are generally undamaged.

Individuals who suffer from conductive hearing loss are typically not considered to be candidates for a cochlear implant due to the irreversible nature of the cochlear implant. Specifically, insertion of the electrode array into a recipient's cochlea results in the destruction of a majority of hair cells within the cochlea. This results in the loss of residual hearing by the recipient.

Rather, individuals suffering from conductive hearing loss typically receive an acoustic hearing aid, referred to as a hearing aid herein. Hearing aids rely on principles of air conduction to transmit acoustic signals through the outer and middle ears to the cochlea. In particular, a hearing aid typically uses an arrangement positioned in the recipient's ear canal to amplify a sound received by the outer ear of the recipient. This amplified sound reaches the cochlea and causes motion of the cochlea fluid and stimulation of the cochlea hair cells.

Unfortunately, not all individuals who suffer from conductive hearing loss are able to derive suitable benefit from hearing aids. For example, some individuals are prone to chronic inflammation or infection of the ear canal and cannot wear hearing aids. Other individuals have malformed or absent outer ear and/or ear canals as a result of a birth defect, or as a result of common medical conditions such as Treacher Collins syndrome or Microtia. Furthermore, hearing aids are typically unsuitable for individuals who suffer from single-sided deafness (total hearing loss only in one ear) or individuals who suffer from mixed hearing losses (i.e., combinations of sensorineural and conductive hearing loss).

When an individual having fully functioning hearing receives an input sound, the sound is transmitted to the cochlea via two primary mechanisms: air conduction and bone conduction. As noted above, hearing aids rely primarily on the principles of air conduction. In contrast, other devices, referred to as bone conduction devices, rely predominantly on vibration of the bones of the recipients skull to provide acoustic signals to the cochlea.

Those individuals who cannot derive suitable benefit from hearing aids may benefit from bone conduction devices. Bone conduction devices convert a received sound into a mechanical vibration representative of the received sound. This vibration is then transferred to the bone structure of the skull, causing vibration of the recipient's skull. This skull vibration results in motion of the fluid of the cochlea. Hair cells inside the cochlea are responsive to this motion of the cochlea fluid, generating nerve impulses resulting in the perception of the received sound.

### SUMMARY

In one aspect of the invention, a bone conduction device for enhancing the hearing of a recipient is provided. The bone conduction device comprises a first sound input device configured to receive acoustic sound signals and generate a first electrical signal representative of the acoustic signal, a second sound input device configured to receive acoustic sound signals and generate a second electrical signal representative of the acoustic signal, electronic circuitry configured to select at least one of the first electrical signal and the second electrical signal, and an electronics module configured to generate a third electrical signal representing the acoustic sound signals based on at least of the first electrical signal and the second electrical signal.

In a second aspect of the present invention, a bone conduction device for enhancing the hearing of a recipient in provided. The bone conduction device, comprises a plurality of sound input elements, each sound input element configured to receive an acoustic sound signal and convert the acoustic signal into an electrical signal, resulting in a plurality of electrical signals, and a switching circuit configured to select at least one of the plurality of electrical signals based on the content of each of the plurality of electronic signals.

In a third aspect of the present invention, a system for enhancing the hearing of a recipient through bone conduction for enhancing the hearing of a recipient in provided. The system comprises an abutment that it is attached to the recipient, the abutment having a recess thereon, a hearing device body portion, the hearing device body portion including, a first microphone configured to receive acoustic sound signals and generate a first electrical signal representative of the acoustic signal, a second microphone configured to receive acoustic sound signals and generate a second electrical signal representative of the acoustic signal, the first and second microphones being substantially equidistant from the longitudinal axis of the device, a switching device configured to select at least one of the first and second electrical signals, and an electronics module configured to generate a third electrical signal representing at least one of the first and second electrical signals, and a coupling member attached to the hearing device body portion, the coupling member having a protrusion thereon and configured to releasably couple to the abutment, wherein when the coupling device is coupled to the abutment, the protrusion engages the recess, thereby selecting one of the first microphone and the second microphones.

Other objects, features and advantages of the present invention will become apparent to those skilled in the art from the following drawings and detailed description of the preferred embodiments.

### BRIEF DESCRIPTION OF THE DRAWINGS

Illustrative embodiments of the present invention are described herein with reference to the accompanying drawings, in which:

FIG. 1 is a perspective view of an exemplary medical device, namely a bone conduction device, in which embodiments of the present invention may be advantageously implemented;

FIG. 2A is a high-level functional block diagram of a bone conduction device, such as the bone conduction device of FIG. 1, in accordance with an embodiment of the invention;

FIG. 2B is detailed functional block diagram of the bone conduction device illustrated in FIG. 2A, in accordance with an embodiment of the invention;

FIG. 3 is an exploded view of an embodiment of a bone conduction device in accordance with one embodiment of FIG. 2B;

FIG. 4 is a view in section of a switching device for selection of a sound input device, in accordance with an embodiment of the invention; and

FIG. 5 is a flowchart illustrating the conversion of an input sound into skull vibration, in accordance with an embodiment of the invention.

### DETAILED DESCRIPTION

Embodiments of the present invention are generally directed to a bone conduction device for converting a received sound signal into a mechanical force for delivery to a recipient's skull. The bone conduction device includes a plurality of sound input components, such as a plurality of microphones, to receive sound signals. The bone conduction device may then select from amongst these received sound signals or combine one or more of the sound signals. The resulting signal (e.g., the selected or combined signal) may then be provided to the recipient so that they may hear the sound corresponding to the resulting signal.

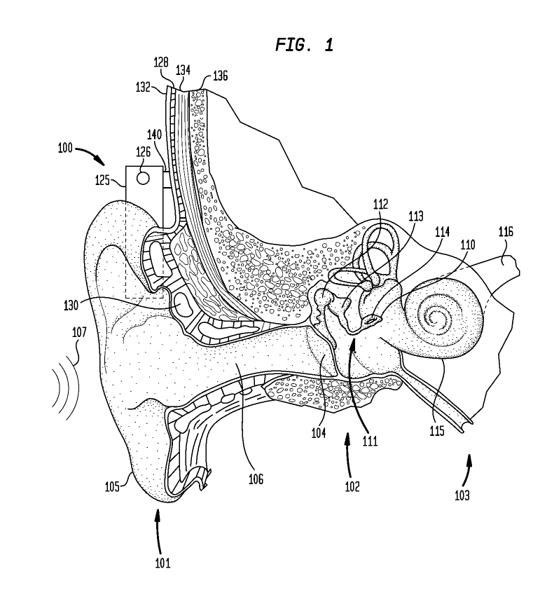

FIG. 1 is a cross sectional view of a human ear and surrounding area, along with a side view of one of the embodiments of a bone conduction device 100. In a fully functional human hearing anatomy, outer ear 101 comprises an auricle 105 and an ear canal 106. A sound wave or acoustic pressure 107 is collected by auricle 105 and channeled into and through ear canal 106. Disposed across the distal end of ear canal 106 is a tympanic membrane 104 which vibrates in response to acoustic wave 107. This vibration is coupled to oval window or fenestra ovalis 110 through three bones of middle ear 102, collectively referred to as the ossicles 111 and comprising the malleus 112, the incus 113 and the stapes 114. Bones 112, 113 and 114 of middle ear 102 serve to filter and amplify acoustic wave 107, causing oval window 110 to articulate, or vibrate. Such vibration sets up waves of fluid motion within cochlea 115. The motion, in turn, activates tiny hair cells (not shown) that line the inside of cochlea 115. Activation of the hair cells causes appropriate nerve impulses to be transferred through the spiral ganglion cells and auditory nerve 116 to the brain (not shown), where they are perceived as sound.

FIG. 1 also illustrates the positioning of bone conduction device 100 relative to outer ear 101, middle ear 102 and inner ear 103 of a recipient of device 100. As shown, bone conduction device 100 may be positioned behind outer ear 101 of the recipient; however it is noted that device 100 may be positioned in any suitable manner.

In the embodiments illustrated in FIG. 1, bone conduction device 100 comprises a housing 125 having a plurality of microphones positioned therein or thereon (in this figure only one microphone 126 is visible). Housing 125 is coupled to the body of the recipient via coupling 140. As described below, bone conduction device 100 may comprise a signal processor, a transducer, transducer drive components and/or various other electronic circuits/devices.

In accordance with embodiments of the present invention, an anchor system (not shown) may be implanted in the recipient. As described below, the anchor system may be fixed to bone 136. In various embodiments, the anchor system may be implanted under skin 132 within muscle 134 and/or fat 128 or the hearing device may be anchored in another suitable manner. In certain embodiments, a coupling 140 attaches device 100 to the anchor system.

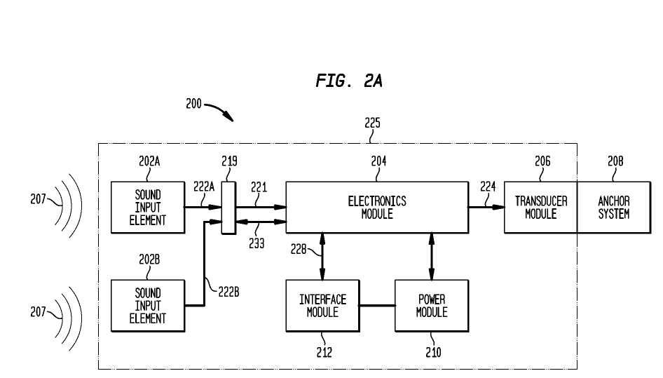

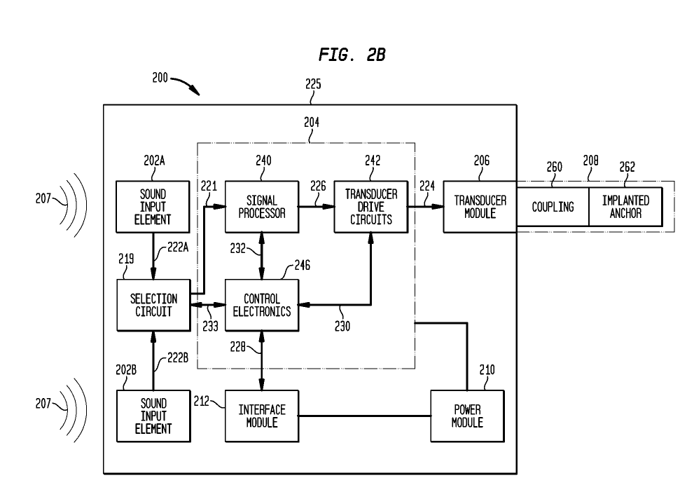

A functional block diagram of one embodiment of bone conduction device 100, referred to as bone conduction device 200, is shown in FIG. 2A. In the illustrated embodiment, a sound 207 is received by sound input elements 202a and 202b, which may be, for example, microphones configured to receive sound 207, and to convert sound 207 into an electrical signal 222. Or, for example, one or more of the sound input elements 202a and 202b might be an interface that the recipient may connect to a sound source, such as for example a jack for receiving a plug that connects to a headphone jack of a portable music player (e.g., MP3 player) or cell phone. It should be noted that these are but some exemplary sound input elements, and the sound input elements may be any component or device capable of providing a signal regarding a sound. Although bone conduction device 200 is illustrated as including two sound input elements 202a and 202b, in other embodiments, bone conduction device 200 may comprise 3 or more sound input elements.

As shown in FIG. 2A, electrical signals 222a and 222b are output by sound input elements 202a and 202b, respectively, to a sound input element selection circuit 219 that selects the sound input element or elements to be used. Selection circuit 219 thus outputs a selected signal 221 that may be electrical signal 222a, 222b, or a combination thereof As discussed below, the selection circuit 219 may select the electrical signal(s) based on, for example, input from the recipient, automatically via a switch, the environment, and/or a sensor in the device, or a combination thereof Additionally, in embodiments, the sound input elements 202 in addition to sending information regarding sound 207 may also transmit information indicative of the position of the sound input element 202 (e.g., its location in the bone conduction device 200) in electrical signal 222.

The selected signal 221 is output to an electronics module 204. Electronics module 204 is configured to convert electrical signals 221 into an adjusted electrical signal 224. Further, electronics module 204 may send control information via control signal 233 to the input selection circuit, such as, for example, information instructing which input sound element(s) should be used or information instructing the input selection circuit 219 to combine the signals 222a and 222b in a particular manner. It should be noted that although in FIG. 2A, the electronics module 204 and input element selection circuit 219 are illustrated as separate functional blocks, in other embodiments, the electronics module 204 may include the input element selection circuit 219. As described below in more detail, electronics module 204 may include a signal processor, control electronics, transducer drive components, and a variety of other elements.

As shown in FIG. 2A, a transducer 206 receives adjusted electrical signal 224 and generates a mechanical output force that is delivered to the skull of the recipient via an anchor system 208 coupled to bone conduction device 200. Delivery of this output force causes one or more of motion or vibration of the recipient's skull, thereby activating the hair cells in the cochlea via cochlea fluid motion.

FIG. 2A also illustrates a power module 210. Power module 210 provides electrical power to one or more components of bone conduction device 200. For ease of illustration, power module 210 has been shown connected only to interface module 212 and electronics module 204. However, it should be appreciated that power module 210 may be used to supply power to any electrically powered circuits/components of bone conduction device 200.

Bone conduction device 200 further includes an interface module 212 that allows the recipient to interact with device 200. For example, interface module 212 may allow the recipient to adjust the volume, alter the speech processing strategies, power on/off the device, etc. Interface module 212 communicates with electronics module 204 via signal line 228.

In the embodiment illustrated in FIG. 2A, sound input elements 202a and 202b, electronics module 204, transducer 206, power module 210 and interface module 212 have all been shown as integrated in a single housing, referred to as housing 225. However, it should be appreciated that in certain embodiments, one or more of the illustrated components may be housed in separate or different housings. Similarly, it should also be appreciated that in such embodiments, direct connections between the various modules and devices are not necessary and that the components may communicate, for example, via wireless connections.

FIG. 2B provides a more detailed functional diagram of bone conduction device 200 of FIG. 2A. In the illustrated embodiment, electronics module 204 comprises a sound or signal processor 240, transducer drive components 242 and control electronics 246. As explained above, in certain embodiments sound input elements 202a and 202b comprise microphones configured to convert a received acoustic signal into electrical signals 222a and 222b.

As illustrated in FIG. 2B, electrical signals 222a and 222b are output from sound input elements 202a and 202b to sound input selection circuit 219. The selection circuit may output electrical signal 221 to signal processor 240. In one embodiment, the selection circuit is a two way switch that is activated by the recipient; however, it is noted that the selection switch may be any switch for operating a plurality of sound input elements, as discussed below. Further, selection circuit 219 may comprise a processor and other components, such that selection circuit 219 may implement a particular combination strategy for combining one or more signals from the sound input elements.

Signal 221 may be signal 222a, 222b or a combination thereof Signal processor 240 uses one or more of a plurality of techniques to selectively process, amplify and/or filter electrical signal 221 to generate a processed signal 226. In certain embodiments, signal processor 240 may comprise substantially the same signal processor as is used in an air conduction hearing aid. In further embodiments, signal processor 240 comprises a digital signal processor.

Processed signal 226 is provided to transducer drive components 242. Transducer drive components 242 output a drive signal 224, to transducer 206. Based on drive signal 224, transducer 206 provides an output force to the skull of the recipient.

For ease of description the electrical signal supplied by transducer drive components 242 to transducer 206 has been referred to as drive signal 224. However, it should be appreciated that processed signal 224 may comprise an unmodified version of processed signal 226.

As noted above, transducer 206 generates an output force to the skull of the recipient via anchor system 208. As shown in FIG. 2B, anchor system 208 comprises a coupling 260 and an implanted anchor 262. Coupling 260 may be attached to one or more of transducer 206 or housing 225. For example, in certain embodiments, coupling 260 is attached to transducer 206 and vibration is applied directly thereto. In other embodiments, coupling 260 is attached to housing 225 and vibration is applied from transducer 206 through housing 225.

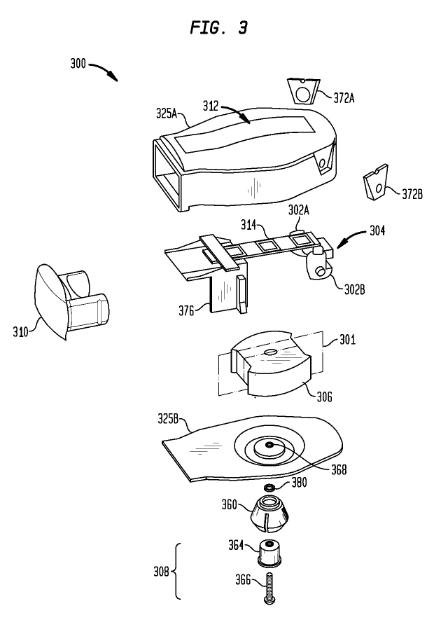

As shown in FIG. 2B, coupling 260 is coupled to an anchor implanted in the recipient, referred to as implanted anchor 262. As explained with reference to FIG. 3, implanted anchor 262 provides an element that transfers the vibration from coupling 260 to the skull of the recipient.

As noted above, a recipient may control various functions of the device via interface module 212. Interface module 212 may include one or more components that allow the recipient to provide inputs to, or receive information from, elements of bone conduction device 200, such, as for example, one or more buttons, dials, display screens, processors, interfaces, etc.

As shown, control electronics 246 may be connected to one or more of interface module 212 via control line 228, signal processor 240 via control line 232, sound input selection circuit 219 via control line 233, and/or transducer drive components 242 via control line 230. In embodiments of the present invention, based on inputs received at interface module 212, control electronics 246 may provide instructions to, or request information from, other components of bone conduction device 200. In certain embodiments, in the absence of recipient inputs, control electronics 246 control the operation of bone conduction device 200.

FIG. 3 illustrates an exploded view of one embodiment of bone conduction device 200 of FIGS. 2A and 2B, referred to herein as bone conduction device 300. As shown, bone conduction device 300 comprises an embodiment of electronics module 204, referred to as electronics module 304. As illustrated, electronics module 304 includes a printed circuit board 314 (PCB) to electrically connect and mechanically support the components of electronics module 304. Further, as explained above, electronics module 304 may also include a signal processor, transducer drive components and control electronics. For ease of illustration, these components have not been illustrated in FIG. 3.

A plurality of sound input elements are attached to PCB 314, shown as microphones 302a and 302b to receive a sound. As illustrated, the two microphones 302a and 302b are positioned equidistant or substantially equidistant from the longitudinal axis of the device; however, in other embodiments microphones 302a and 302b may be positioned in any suitable position. By being positioned equidistant or substantially equidistant from the longitudinal axis, bone conduction device 300 can be used on either side of a patient's head. The microphone facing the front of the recipient is generally chosen using the selection circuit as the operating microphone, so that sounds in front of the recipient can be heard; however, the microphone facing the rear of the recipient can be chosen, if desired.

Bone conduction device 300 further comprises a battery shoe 310 for supplying power to components of device 300. Battery shoe 310 may include one or more batteries. As shown, PCB 314 is attached to a connector 376 configured to mate with battery shoe 310. Connector 376 and battery shoe 310 may be, for example, configured to releasably snap-lock to one another. Additionally, one or more battery connects (not shown) are disposed in connector 376 to electrically connect battery shoe 310 with electronics module 304.

In the embodiment illustrated in FIG. 3, bone conduction device 300 further includes a two-part housing 325, comprising first housing portion 325a and second housing portion 325b. Housing portions 325 are configured to mate with one another to substantially seal bone conduction device 300.

In the embodiment of FIG. 3, first housing portion 325a includes an opening for receiving battery shoe 310. This opening may be used to permit battery shoe 310 to inserted or removed by the recipient through the opening into/from connector 376. Also in the illustrated embodiment, microphone covers 372 can be releasably attached to first housing portion 325a. Microphone covers 372 can provide a barrier over microphones 302 to protect microphones 302 from dust, dirt or other debris.

Bone conduction device 300 further may include an embodiment of interface module 212, referred to in FIG. 3 as interface module 312. Interface module 312 is configured to provide information or receive user input from the user.

Also as shown in FIG. 3, bone conduction device 300 may comprise a transducer 206, referred to as transducer 306, and an anchor system 208, referred to as anchor system 308 in FIG. 3. As noted above, transducer 306 may be used to generate an output force using anchor system 308 that causes movement of the cochlea fluid to enable sound to be perceived by the recipient. The output force may result in mechanical vibration of the recipient's skull, or in physical movement of the skull about the neck of the recipient. Anchor system 308 comprises a coupling 360 and implanted anchor 362. Coupling 360 may be configured to attach to second housing portion 325b. As such, vibration from transducer 306 may be provided to coupling 360 through housing 325b. As illustrated, housing portion 325b may include an opening to allow a screw (not shown) to be inserted through opening 368 to attach transducer 306 to coupling 360. In such embodiments, an O-ring 380 may be provided to seal opening 368 around the screw.

As noted above, anchor system 308 includes implanted anchor 362. Implanted anchor 362 comprises a bone screw 366 implanted in the skull of the recipient and an abutment 364. In an implanted configuration, screw 366 protrudes from the recipient's skull through the skin. Abutment 364 is attached to screw 366 above the recipient's skin. In other embodiments, abutment 364 and screw 366 may be integrated into a single implantable component. Coupling 360 is configured to be releasably attached to abutment 364 to create a vibratory pathway between transducer 306 and the skull of the recipient. Using coupling 360, the recipient may releasably detach the bone conduction device 300 from anchor system 308. The user may then make adjustments to the bone conduction device 300 using interface module 312, and when finished reattach the bone conduction device 300 to anchor system 308 using coupling 360. A further description of exemplary user interface modules 312 and how they may be used by a user to view data or adjust control settings of the hearing device is provided in the U.S. patent application by John Parker, Christian Peclat, and Christoph Kissling entitled “A Bone Conduction Device with a User Interface,” filed concurrent with the present application, which is incorporated by reference herein in its entirety.

As noted above, bone conduction device 300 may comprise two or more sound input elements, such as microphones 302a and 302b. Referring back to FIG. 2B, these microphones may be represented as sound input elements 202a and 202b. Further, as previously noted, a selection circuit 219 may be used to select from different input elements 202a and 202b or combine the signals from the input elements 202a and 202b in some manner. In an embodiment the recipient may use a user interface 212 of the hearing device 200 to select from amongst the different input elements or direct the hearing device to implement a particular strategy to combine or select the signals from the input elements 202a and 202b.

One exemplary combining strategy is for the recipient, though the user interface, to selectively chose one of the microphones to function as a dominant microphone. If a microphone is selected to be the dominant microphone, then the signal processor may select and use the dominant signal and disregard the other signals in the event certain conditions arise, such as if the signal processor receives multiple noisy signals from each of the microphones and the signal processor is unable to determine which microphone signal includes the sound that would be of principal interest to the recipient. Similarly, in certain embodiments, the recipient may use the user interface to select an order of dominance for the microphones, such that, in noisy conditions, the signal processor first tries to decode the primary dominant microphone signal. If, however, the signal processor determines that this decoding fails to meet certain conditions (e.g., it appear to be noise), the signal processor then selects the next most dominant microphone signal. The signal processor may then, for example, continue selecting and decoding signals using this order of dominance until a microphone signal is decoded that meets specified conditions (e.g., the signal appears to include speech or music). It should be noted, however, that these are merely exemplary strategies that may be employed for selecting amongst multiple microphone signals, and in other embodiments other strategies may be used.

Another exemplary combining strategy that may be employed is for the hearing device 200 to use a weighting system. For example, the signal processor 240 may instruct the selection circuit 219 to individually weight the different signals and then combine the weighted signals. This may be accomplished, for example, by the selection circuit applying fixed weights (e.g., weights specified by the recipient using the user interface or a strategy that weights signals from more forward facing sound elements higher) to each of the signals. Or, for example, the selection circuit 219 may examine each of the input signals and then weight the signals based on this analysis. One exemplary strategy for analyzing the signals is for the selection circuit 219 to examine each signal to determine if the signal appears to include speech information. If so, the selection circuit 219 may give a higher weight to the signal, while providing a lower weight to signals with little to no speech. Similarly, this strategy may also take into account the location of the sound input element 202. For example, the hearing device 200 may be configured to more heavily weight signals from forward facing sound input elements 202 than from rear facing sound elements, even if both are determined to include speech information. This may be useful because in crowded rooms it is more likely that the recipient will be speaking with someone they are facing than someone behind them.

In yet another exemplary combining strategy, the hearing device 200 may permit the recipient, via the user interface, to select a control setting that turns on a direction finding algorithm for selecting between microphones. Such algorithms are known to one of ordinary skill in the art. For example, simultaneous phase information from each receiver may be used to estimate the angle-of-arrival of the sound. Using such algorithms, the signal processor may determine a suitable microphone output signal or a plurality of suitable microphone outputs to use in providing the sound to the recipient.

It should be noted that these are but some exemplary combination strategies that a bone conduction device may be able to use in combining signals from a plurality of sound input elements, and in other embodiments other strategies may be used. Additionally, although the embodiments are discussed with reference to the recipient selecting the combining strategy, it should be understood that any user (e.g., the recipient, a doctor, a family member, friend, etc.) may make these selections. Or, for example, a particular combining strategy may be fixed in hardware or software of the hearing device. Further, as discussed above, in embodiments, the recipient may be able to use a user interface 212 for the hearing device 200 to select and combination strategy to be used, such as the above referenced U.S. patent application by John Parker, Christian Peclat, and Christoph Kissling, entitled “A Bone Conduction Device with a User Interface.”

As noted above in certain embodiments, the hearing device may select and use only signals from the forward facing sound input element(s). Or, for example, the hearing device may weight signals from forward facing sound input elements higher than rear facing sound input elements. Further, in certain embodiments the anchor system for the hearing device may implanted on either the right or left ear of a recipient. For example, a doctor may wish to implant the hearing device's anchor system on the side of the recipients head that the doctor believes will provide the recipient with the best hearing. Thus, doctors would like the flexibility to install anchor systems on either the left or right side of a recipients head. Accordingly, hearing devices in accordance with embodiments of the present invention may be configured so that the hearing device may be used both with anchor systems implanted on the right side and left side of a recipients head. However, because the hearing device may be implanted on either side of a recipients head, it may not be able to tell during manufacture of the hearing device which microphone(s) will be forward facing and which microphone(s) will not be forward facing. The following disclosure provides a description of an exemplary mechanism that a hearing device may employ to determine the forward facing microphone(s).

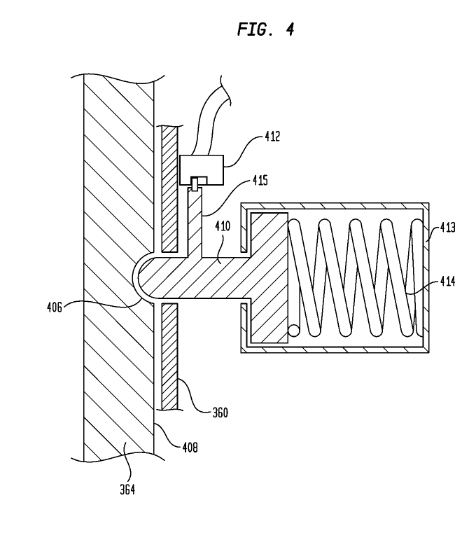

FIG. 4 illustrates a close-up view of an exemplary mechanism that a hearing device may use to determine whether it is attached on the left or right side of a recipient. This exemplary mechanism may be used with a hearing device such as bone conduction device 300 illustrated in the above-discussed FIG. 3. This exemplary mechanism uses two different types of abutments 364, one type for each side of the head. The different types of abutments (i.e., the left and right types) may be marked with words (e.g., “Right” and “Left”), use unique colors, or use some other mechanism to help a doctor quickly identify the type of abutment. Further, the different abutment types (i.e., left and right) may have a slightly different shape that may be detectable by the bone conduction device 300 so that the hearing device may determine to which side of the recipient the bone conduction device 300 has been attached. In the example of FIG. 4, the abutment 364 for one side includes an indentation in the center of its top face (i.e., the face of the abutment that faces the hearing device), while the abutment for the other side does not include such an indentation but instead has a flat surface along its top face. For explanatory purposes, FIG. 4 will be discussed with reference to abutment 364 including an indentation, and this will be assumed in this example to be the left side abutment. In other embodiments, abutment 364 may have another type of recess, such as an opening, or aperture. As used herein the term “recess” refers to any type of indentation, hollow, slit, opening, or aperture.

As illustrated in FIG. 4, the bone conduction device 300 includes a mechanical switch 412. This switch 412 may be installed at any suitable location in the bone conduction device. For example, in an embodiment, switch 412 may be mounted on the inside or outside of second housing portion 325b. Further, switch 412 may be any suitable type of switch, such as, for example, an electronic switch, a mechanical switch, or a magnetic switch. For simplification, second housing portion 325b is not illustrated in FIG. 4 As noted above, in this example, abutment 364 includes an indentation 406 located on the surface 408 of the abutment. This indentation 406 is sized to receives a protrusion 410 (e.g., a pin) from the bone conduction device 300. Thus, if the protrusion 410 fits within the indentation 406, bone conduction device 300 will know it is on the left side, while if there is no indentation then the protrusion will not be able to extend into the abutment 364 and the bone conduction device will know that it is located on the right side of the recipient. Protrusion 410 may be include in a spring loaded housing 413 that may be mounted, for example, on the inside or outside of second housing portion 325b or any other suitable location. If housing 413 is mounted on the inside of second housing portion 325b, the protrusion 410 may extend through opening 368 in the second housing portion 325b and into coupling device 360 so that protrusion 410 will fit in indentation 406 when the bone conduction device 300 is attached to abutment 364. The protrusion housing may include a spring 414, such that when there is no indentation in the abutment 364, the protrusion is pushed back, while if there is an indentation 406, the spring 414 pushes protrusion 410 into the indentation.

Protrusion 410 may further include an arm member 415 that will contact switch 412 when protrusion 410 fits in indentation 406 but will not contact switch 412 when abutment 364 does not have an indentation and protrusion 410 (and accordingly its arm member 415) are thus pushed back towards the protrusion housing 413. Thus, in this example, switch 412 determines that the bone conduction device 300 is attached to the left side if the switch 412 is contacted by the arm member 415, and determines that the bone conduction device 300 is attached to right side if arm member 415 is not in contact with the switch 412. Switch 412 may then send an indication to, for example, the signal processor of the bone conduction device 300 that indicates which side the bone conduction device 300 is attached. Or, for example, the switch 412 may simply send a signal indicating whether the arm member 415 is touching the switch (e.g., switch closed) or not (e.g., switch open). The signal processor may store information that specifies whether the bone conduction device 300 is connected to the left or right side of the recipient based on the possible signals from the switch 412. For example, the signal processor may store information that specifies that the signal processor should consider the bone conduction device connected to the left side if it receives a switch closed signal from the switch 412, and should consider the bone conduction device connected to the right side if the signal processor receives a switch open signal from the switch 412.

It should be noted that the embodiment of FIG. 4 is but one exemplary embodiment and in other embodiments other suitable mechanisms may be used for determining to which said of a recipient a bone conduction device is attached. For example, in other embodiments the switch and protrusion may be located in a different location on bone conduction device 300. Or, for example, bone conduction device 300 may instead use an electrical switch, such as a magnetic switch that indicates the presence of a particular magnetic field, and corresponding magnets may be placed in one type of abutment (e.g., for the left side of the recipient) and not included in the other type of abutment (e.g., for the right side).

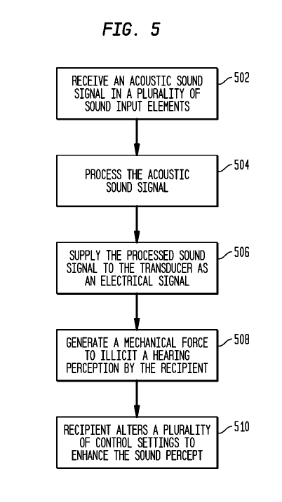

FIG. 5 illustrates the conversion of an input sound signal into a mechanical force for delivery to the recipient's skull in accordance with embodiments of bone conduction device 300. At block 502, bone conduction device 300 receives a sound signal. In certain embodiments, the sound signal is received via microphones 302a and 302b.

At block 504, the signal is selected by the input selection circuit. The sound input selection circuit determines which signal or signals is to be output, based on the manual or automatic settings discussed above.

At block 506, the sound signal received by bone conduction device 300 is processed by the speech processor in electronics module 304. As explained above, the speech processor may be similar to speech processors used in hearing aids. In such embodiments, speech processor may selectively amplify, filter and/or modify sound signal. For example, speech processor may be used to eliminate background or other unwanted noise signals received by bone conduction device 300. In other embodiments, as discussed above, the speech processor may include programming to select a signal or combine signals, resulting in an improved percept by the recipient.

At block 508, the processed sound signal is provided to transducer 306 as an electrical signal. At block 510, transducer 306 converts the electrical signal into a mechanical force configured to be delivered to the recipient's skull via anchor system 308 so as to illicit a hearing perception of the sound signal.

Although the above description was discussed with reference to the recipient using the hearing device, it should be understood that this was provided for explanatory purposes and the hearing device and its user interface may be used in a similar manner by any user (e.g., doctor, family member, friend, or any other person).

Although the present invention has been fully described in conjunction with several embodiments thereof with reference to the accompanying drawings, it is to be understood that various changes and modifications may be apparent to those skilled in the art. Such changes and modifications are to be understood as included within the scope of the present invention as defined by the appended claims, unless they depart there from.

What is claimed is:

1. A bone conduction device for enhancing the hearing of a recipient, comprising:
a first sound input device configured to receive sound signals and generate a first electrical signal representative of said acoustic signal;
a second sound input device configured to receive sound signals and generate a second electrical signal representative of said signal;
electronic circuitry configured to select at least one of the first electrical signal and the second electrical signal; and
an electronics module configured to generate a third electrical signal representing said sound signals based on at least one of said first electrical signal and the second electrical signal.

2. The device of claim 1, wherein the electronic module includes a switch selected from a group consisting of a mechanical switch, a magnetic switch or an electrical switch.

3. The device of claim 1, wherein the first sound input device and the second sound input device are positioned substantially equidistant from the longitudinal axis of the device.

4. The device of claim 1, wherein the electronic circuitry is in communication with a user interface and is configured to select at least one of the first sound input device and the second sound input device based on recipient input into the user interface.

5. The device of claim 1, wherein the electronic circuitry is configured to allow the recipient to select one of the first sound input device and the second sound input device as the dominant sound input device through the user interface.

6. The device of claim 1, wherein the electronic circuitry is configured to select at least one of the first sound input device and the second sound input device based on the first and second electrical signals sent from the first sound input device and the second sound input device.

7. The device of claim 1, wherein the first and second electrical signals include information indicating the position of the respective sound input device relative the recipient.

8. The device of claim 1, further comprising
an abutment that it is attached to the recipient, the abutment having an recess thereon; and
a coupling member attached to the device, the coupling member configured to releasably couple to the abutment and having a protrusion therein;
wherein when the coupling device is coupled to the abutment, the protrusion engages the recess, thereby activating a switch that sends a selection signal to the electronic circuitry, selecting one of the first sound input device and the second sound input device.

9. The device of claim 1, wherein
the first and second sound input devices are microphones.

10. The device of claim 1, wherein
the electronic circuitry utilizes a direction finding algorithm to select at least one of the first sound input device and the second sound input device.

11. A bone conduction device for enhancing the hearing of a recipient, comprising:
a plurality of sound input elements, each sound input element configured to receive an sound signal and convert the signal into an electrical signal, resulting in a plurality of electrical signals; and
a switching circuit configured to select at least one of the plurality of electrical signals based on the content of each of the plurality of electronic signals.

12. The device of claim 11, wherein at least one of the sound input elements is positioned substantially equidistant from the longitudinal axis of the bone conduction device as at least one other sound input element.

13. The device of claim 11, wherein the switching circuit is configured to override selection of at least one of the first input element and the second input element based on recipient input.

14. The device of claim 11, wherein each of the plurality of electrical signals includes information regarding the position of the each of the plurality of input elements.

15. The device of claim 11, wherein the plurality of sound input elements are microphones.

16. The device of claim 11, wherein the switching circuit utilizes a direction finding algorithm to select at least one of the plurality of electrical signals.

17. The device of claim 11, wherein the switching circuit is configured to allow the recipient to select one of the plurality of input devices as the dominant input device through a user interface.

18. The device of claim 11, wherein the switching circuit is configured to select at least one of the plurality of electrical signals based on the strength of the signal.

19. A system for enhancing the hearing of a recipient through bone conduction, comprising:
an abutment that it is attached to the recipient, the abutment having a recess thereon;
a hearing device body portion, the hearing device body portion including, a first microphone configured to receive sound signals and generate a first electrical signal representative of said signal, a second microphone configured to receive sound signals and generate a second electrical signal representative of said signal, said first and second microphones being substantially equidistant from the longitudinal axis of the device, a switching device configured to select at least one of the first and second electrical signals, and an electronics module configured to generate a third electrical signal representing at least one of said first and second electrical signals; and
a coupling member attached to the hearing device body portion, the coupling member having a protrusion therein and configured to releasably couple to the abutment;
wherein when the coupling device is coupled to the abutment, the protrusion engages the recess, thereby selecting one of the first microphone and the second microphones.

20. A The system of claim 19, further comprising
a two mode switch;
wherein when the switch is in a first mode the first microphone is selected and when the switch is in a second mode the second microphone is selected.
